(19) United States
(12) Patent Application Publication
(10) Pub. No.: US 2008/0208172 A1
(43) Pub. Date: Aug. 28, 2008

(54) Colostomy Pump System

(76) Inventors: Steven John Marshall, New South Wales (AU); Matthew Campbell Greaves, Queensland (AU); Rodney Gordon Parkin, New South Wales (AU); Peter Davekumar Stephens, New South Wales (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/794,292

(22) PCT Filed: Jan. 19, 2006
(86) PCT No.: PCT/AU06/00057

§ 371 Date: Mar. 27, 2008

(30) Foreign Application Priority Data
Jan. 19, 2005 (AU) 2005900378

(51) Int. Cl. A61M 1/00; F04B 43/12

(52) U.S. Cl. 604/540; 417/476

(57) ABSTRACT

A waste evacuator device (10) for evacuating waste products from bodily orifices, including stomas and artificial stomas created as a result of a surgical procedure such as a colostomy is disclosed. The device comprises a chamber (11) having an inlet (14) which is fluidly communicable with an orifice formed in a mammalian body. An irrigation member (12) is provided for introducing irrigating fluid into the orifice. A suctioning device (13) connectable to the chamber to create a region of reduced pressure within the chamber. An irrigation and evacuation control means for the device is provided for receiving signals generated by at least one sensor, the signals being representative of at least the performance of the irrigation member and of the suctioning device.

### CROSS-REFERENCE TO RELATED APPLICATIONS

The present application claims priority from Australian Provisional Patent 2005900378 filed on 19 Jan. 2005, the content of which is incorporated herein by reference.

### FIELD OF THE INVENTION

The present invention relates to a device, system and method for evacuating waste product from bodily orifices, including stomas and in particular artificial stomas created as a result of surgical procedures, such as colostomies.

### BACKGROUND TO THE INVENTION

Patient's suffering from bowel disease, congenital abnormality or bowel injury may not have adequate use of their bowel to evacuate waste. In such cases, an artificial stoma is treated to allow waste evacuation.

Faecal matter is typically collected in a bag that is held against or stuck to the skin of the abdomen around the stoma.

The use of collection bags imposes several problems for the ostomate, including restrictions to lifestyle (eg. poor utility, inconvenience, and need for dietary changes), psychological issues (eg. sexuality and poor self esteem) and an increased risk of herniation.

The introduction of irrigating liquid into the large intestine through the stoma may be used to stimulate faecal evacuation prior to collection. This process relies upon the force of gravity to introduce the irrigation fluid into the bowel. Distension of the bowel by the irrigation fluid causes peristalsis in the colon causing waste material to be ejected from the bowel through the stoma.

The present invention aims to provide a still further means of evacuating waste material which overcomes the problems associated with the traditional bag devices and improves the efficacy of traditional irrigation systems.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated clement, integer or step, or group of elements, integers or steps, but not the exclusion of any other element; integer or step, or group of elements, integers or steps.

According to a first aspect, the present invention consists in a waste evacuator device including:

a chamber having an inlet fluidly communicable with an orifice in a mammalian body;

an irrigation member for introducing irrigating fluid into the orifice;

a suctioning device connectable to the chamber to create a region of reduced pressure within the chamber; and

an irrigation and evacuation control means for the device, said control means receiving signals generated by at least one sensor, said signals being representative of at least both the performance of the irrigation member and of the suctioning device.

In a second aspect, the present invention consists in a system for evacuating waste through an orifice of a mammalian body, the system including:

a chamber having an inlet fluidly communicable with all orifice in a mammalian body;

an irrigation member for introducing irrigating fluid into the orifice;

a suctioning device connectable to the chamber to create a region of reduced pressure within the chamber; and

a control means to at least partially control at least both the introduction of irrigating fluid into the orifice and the reduction of pressure within the chamber.

In a third aspect the present invention consists in a method for evacuating waste through an orifice in a mammalian body, the method including:

(a) providing a device including a chamber, said chamber having an inlet, the device further including an irrigation member for introducing irrigating fluid into the orifice, a suctioning device, connectable to the chamber to create a region of reduced pressure within the chamber; and a control means to control the introduction of irrigating fluid into the orifice and the reduction of pressure within the chamber;

(b) bringing the inlet of the device into fluid communication with the orifice;

(c) introducing irrigating fluid into the orifice trough the irrigation member wherein the introduction of the irrigating fluid is at least partially controlled by the control means of the device;

(d) applying suction to draw the waste material from the body and into the chamber of the device wherein the section is at least partially controlled by the control means of the device.

The chamber of the device typically has an inlet. The inlet may abut with the skin of a patient around the orifice. The chamber also has an outlet that is connectable to the suctioning device of the device.

In one embodiment, the inlet of the chamber seals with the body about the stoma. The inlet can be formed from a resiliently flexible material or a rigid material. For example, the inlet can be formed from a polymeric or elastomeric material. In another embodiment, the inlet can be substantially circular, however, other inlet shapes can be readily envisaged. The inlet is preferably located at a first end of the chamber.

The outlet of the chamber is preferably located at a second end of the chamber distal the first end. The outlet preferably comprises an opening in the second end of the chamber. The outlet is typically connected to the suctioning device by tubing wherein said tubing has a sufficient diameter to accommodate the waste matter drawn from the body.

In one embodiment, the chamber has a longitudinal axis and is symmetrical or asymmetrical about this axis. The diameter of the chamber may decrease from the first end to the second end. In other embodiments, it can be envisaged that the chamber might increase in diameter from the first end to the second end or may be of a constant diameter along all or a portion of its length.

In one embodiment, the chamber of the device is angled along the longitudinal axis such that the outlet is below the inlet when the device is in use. This embodiment uses the force of gravity to aid the removal of waste material from the chamber.

The chamber may be formed from any suitable material. In one embodiment, the chamber can be formed from a readily cleanable material. The chamber may be formed from a semitransparent material to enable a user to watch the passage of the waste products through the chamber. As an example only, the chamber may be formed from a transparent polymethylmethacrylate (PMMA) material.

The irrigation member may comprise a catheter having at least one lumen extending therethrough for the passage of irrigating fluid. The lumen may be in fluid communication with an orifice or a plurality of orifices at or adjacent, a free end of the catheter.

The catheter typically extends into the chamber through an opening in a sidewall of the chamber or, alternatively, through the second end of the chamber. The free end of the catheter is positionable within the chamber. The catheter is connected to a fluid source at an opposite end to the free end.

The catheter is typically movable relative to the chamber such that its free end is moveable between a first position within the chamber and a second position extending beyond the inlet of the chamber. When the device is in use and, particularly during the irrigation process, the catheter is moved to the second position such that the free end is insertable into the orifice. In the first position, that is, after irrigation, the free end of the catheter may be withdrawn back into the chamber and preferably to a position approximately 30-nm from the inlet. This prevents interference by the catheter with the subsequent evacuation process

The catheter may be resiliently flexible along all or a portion of its length. Alternatively, a length of the catheter may be relatively rigid with only a tip portion at, or adjacent, the free end comprising a relatively more flexible material.

In one embodiment, the catheter includes an irrigation cone at, or adjacent, the free end. The irrigation cone may be a separate component or may form part of the irrigation catheter. The irrigation cone comprises a tip portion and a skirt portion depending from the tip portion. The skirt portion typically increases in diameter away from the tip portion. The skirt portion is engageable with the skin of a patient around the stoma of a user to substantially seal the stoma. The tip portion may be made from the same material as the skirt portion or a different material. For example, the tip portion may be made from a relatively more rigid material. The skirt portion is typically integrally formed with the tip portion.

The irrigation cone may further include a neck portion. The irrigation cone is typically connected to the remainder of the catheter by said neck portion. The neck portion is preferably made from a suitably flexible to allow the irrigation cone to pivot relative to the catheter. This embodiment advantageously enables use with any shape or configuration of stoma. For example, if the bowel of an ostomate is joined at an acute angle to the stoma, a straight or fixed irrigation cone will not fit readily into the intestine. The pivotability allowed by the flexible neck portion allows the irrigation cone of this embodiment to conform with any number of configurations of the bowel/stoma.

The irrigating fluid is typically water or a mixture including water. Stool softening agents such as liquid paraffin could be utilised.

The irrigating fluid is typically forced through the catheter lumen by an irrigation pump. The pressure created by the pump to allow the flow of the irrigating fluid through the catheter lumen may be varied by the control means of the device but typically does not exceed 140 cmH₂O. The control means way vary the pressure over the range of from 50 cmH₂O to 140 cmH₂O.

The flow of irrigating fluid through the catheter lumen may be controlled by the pumping pressure and may be increased or decreased during the irrigation process. Typically, the irrigating fluid is not introduced at a flow rate of greater than 10 ml/sec.

The control means of the device may also control the volume of fluid introduced in addition to controlling the flow rate at which it is introduced.

The control means may receive signals indicative of the volume of irrigating fluid and the flow rate (including the pressure created by the irrigation pump) and can generate output control signals to modify the irrigation cycle. The control means will be discussed in further detail below.

The irrigation pump further provides a continuous positive pressure in the lumen of the catheter after the irrigation process has ceased. This prevents any waste material entering the lumen during the subsequent evacuation process.

The irrigating fluid is typically held in an irrigation reservoir of the device. The reservoir may be purely for the storage of irrigating fluid or, alternatively, the reservoir may contain extra fluid for assisting the evacuation process and for washing the device.

Typically, the irrigation reservoir has at least a 2 litre capacity if used to store irrigation fluid alone. The volume of irrigating fluid delivered to a patient may be varied to suit an individual patient's needs. The volume of fluid delivered may range from around 250 ml to around 1500 ml and more preferably from around 400 ml to around 1000 ml and more preferably still from around 500 ml to around 700 ml.

As described above, the volume of fluid delivered to the patient may be controlled by the control means. In one embodiment, the control means may receive signals from a sensor in the irrigation reservoir. The sensor may be a pressure sensor with the signals generated and processed by the control means indicative of the volume of irrigating fluid that has been delivered to the patient. When the pressure reaches a pre-determined level, that is, when a predetermined volume of fluid has been delivered to the patient, the control means may send an output signal to cease the irrigation process.

A patient may also select the volume of irrigating fluid that they desire to be delivered. The volume delivered is then controlled by the control means of the device. As a safety feature, typically the control means has a capability of preventing the introduction of more than approximately 1500 ml of irrigating fluid unless there is specific user intervention.

In the embodiment that the irrigation reservoir is a multi-functional reservoir i.e. also contains fluid for evacuation and washing, said reservoir may have a volume capacity of up to approximately 15 litres.

The evacuation and washing fluid may, alternatively, be contained within a separate reservoir having a capacity of at least 10 litres.

A pressure sensor in the multi-functional reservoir will also communicate signals indicative of the volume of irrigating fluid that has been delivered to the patient to the control means such that the control means can cease emptying of the reservoir and this the irrigation process. In the same manner, the pressure sensor may communicate signals indicative of the volumes of fluid emptied for the evacuation and the washing cycles to allow the control means to control said volumes.

The control means of the present invention typically comprises a micro-processor that can receive electrical signals from sensors located in the device. The micro-processor processes the electrical signals and may generate output signals. This feedback system controls a number of functions of the device including, as mentioned above, the volume and flow rate of irrigating fluid delivered to the patient. The other functions of the control means will be discussed in further detail below

In addition to the microprocessor control means, the device of the present invention may include a number of manual control systems.

In one embodiment, the commencement of irrigation requires a manual input by the user. Temporary cessation and subsequent resumption of the flow of irrigating fluid may be controlled by the microprocessor or, alternatively, by manual input.

Typically, a user may over-ride the microprocessor control. For example, a user may wish to commence evacuation of the bowel before the pre-determined volume of irrigating fluid has been introduced into the bowel. The user may manually cease the irrigation process and commence the evacuation process.

The suctioning device of the present invention may comprise a peristaltic-type pump. The pump typically provides a vacuum pressure ranging from 5 to 140 cmH₂O. The pressure may be varied during the evacuation process as required by the user or by the control means.

The pump may be a dual action pump that also acts as the irrigation pump. The irrigation pump and suctioning device may, however, comprise separate members.

The tubing connected to the outlet of the chamber passes through a housing of the pump. If the pump also functions as the irrigation pump, irrigation tubing connecting the irrigation catheter to a fluid source also passes through the housing of the pumping device.

The pump preferably incorporates one or more rollers adapted to be forced along a length of the tubing passing through the pump and so force the contents of the tubing along that length of tubing. The rollers may be mounted on a disc adapted to rotate with the housing. On rotation of the disc, the rollers compress the tubing that may be mounted between the rollers and an outer wall of the housing. The rotation of the disc is preferably provided by a central or an off-centre drive shaft that is rotated by a motor in the housing. The motor is typically powered by a battery but may be mains powered.

Typically, the rollers of the pump provides sufficient force upon the tubing to crush hard faecal matter that may be passing through the tubing.

The pump may also have a release mechanism to cause some of the rollers to move away from the sidewall of the housing. This embodiment has the advantage of clearing any major blockages in the tubing that may occur, for example, due to the presence of hard material in the bodily waste material. The further advantage of this embodiment is that the tubing may be removed from the housing after use. New tubing may then be placed in the housing for re-use. This is particularly advantageous in cases where the device is used for more than one individual.

The vacuum pressure created by the pump may be limited to an upper level of approximately 140 cmH₂O. As a safety feature, the control means of the device may prevent the pump exceeding this upper level.

In one embodiment, the control means receives signals from a pressure sensor in the chamber or tubing connecting the chamber to the pumping device. If the signals indicate that the pressure has reached the upper level, the control means generates output signals to either temporarily stop the evacuation or to slow the peristaltic pump such that it generates a lesser vacuum pressure.

In addition to the control means, the device may her include a mechanical safety control member to prevent the suction pressure from exceeding the upper level in the event that the control means fails. The safety control member may comprise a release valve in a wall of the chamber.

Commencement, cessation, temporary cessation and resumption of the functions of the device (irrigation, evacuation, washing) may be controlled either automatically by the control means or manually by a user. At all the times the user may have the option of over-riding the control means as required.

During the evacuation of waste product, the irrigation catheter may deliver a steady stream of irrigating fluid to the chamber of the device. This additional fluid assists in the removal of the waste from the chamber. The volume of irrigating fluid delivered to the chamber during the evacuation step may be predetermined or varied 1 during evacuation. The volume and rate of flow of the irrigating fluid into the chamber during evacuation is typically controlled by the control means.

The device may further provide at least one flush cycle to cause an increased volume of fluid to be delivered to the chamber during evacuation. The commencement volume of fluid delivered and cessation of the flush cycle may be controlled by the control means. In this embodiment, the control means may be programmed to control a plurality of flush cycles during the evacuation step. Alternatively, the at least one flush cycle may be controlled by a user. In the latter embodiment, the device may include a “flush button” or lever much as one would find on a standard toilet. Depression of the button or lever may then actuate the flushing cycle(s).

The flush cycle(s) typically provide a temporary increase in irrigating fluid in the chamber. During the flush cycle, the control means may also cause an increase in the speed of the pumping device thus causing an increase in vacuum pressure in the chamber. The increased pressure aids the removal of the extra fluid of the flush cycle.

The device may further comprise a washing function. In this embodiment, fluid is drawn from either a multi-functional irrigation reservoir or a separate washing reservoir. Either reservoir typically has the capacity to hold a volume of approximately 5 to 10 litres of fluid for washing;

If the device includes a separate washing reservoir, the washing fluid held therein may also include cleaning chemicals. Said chemicals are typically not hazardous to a user such that inadvertent use of the washing fluid as irrigating fluid would not be detrimental.

In addition to controlling the volume of washing fluid, the control means of the device may control the commencement, cessation, temporary cessation and resumption of the washing cycle of the device. Particularly, the control means may sense when the reservoir has been emptied and cease the washing cycle. Alternatively, the washing cycle may be manually controlled.

The fluid for the washing cycle is pumped from a multi-functional reservoir or a separate washing fluid reservoir through the parts of the device that have come into contact with the waste material evacuated from the patient. The washing fluid is pumped using the irrigation pump and/or the evacuation pump. The flow rate of the fluid may also be controlled by the control means of the device. Typically the pump pressures employed to wash the device are greater than the pressures required for flushing and evacuation.

When water is used as the fluid for irrigation, evacuation and washing of the device, the water can be supplied from any normal water source, including a mains tap or a portable water container.

The device may also comprise a heating means to warm the water before it is pumped through the catheter lumen during the irrigation cycle. Such a heating means can comprise an electrical heating element in the irrigation reservoir.

In the above aspects, the device and/or system can comprise a portable device/system. The power for the pumping means and/or suction means can be provided by a battery. The battery is preferably rechargeable and portable along with the device. The device is preferably in a case member. The case member is typically mounted on a set of wheels for easy maneuverability. Alternatively, the power may be drawn from the mains supply.

In a fourth aspect, the present invention consists in a waste evacuation device including:

a housing having at least one fluid reservoir therein;

a chamber having an inlet fluidly communicable with an orifice in a mammalian body;

an irrigation member for introducing irrigating fluid into the orifice, said irrigation member connectable to the fluid reservoir; and

a suctioning device connectable to the chamber to create a region of reduced pressure within the chamber;

wherein, when the device is not in use, the chamber, the irrigation member and the suctioning device are housed with the fluid reservoir of the housing

In the fourth aspect, the at least one reservoir may store irrigating fluid when the device is in use. Additionally, the at least one reservoir may store fluid for assisting the evacuation process and for washing the device after use.

The device may comprise more than one reservoir, wherein the chamber, the irrigation member and the suctioning device housed within one of said reservoirs when the device is not in use.

The device of the fourth aspect may comprise separate upper and a lower housings that are connectable to one another. The upper housing may comprise the chamber, the irrigation member and the suctioning device. The lower housing may comprise the at least one reservoir.

The upper housing is movable relative to the lower housing between a storage configuration wherein the upper housing is substantially housed within the lower housing such that the chamber, the irrigation member and the suctioning device are housed within the at least one reservoir, and an active configuration wherein the upper housing extends from the lower housing to expose the chamber, the irrigation member and the suctioning device ready for use. Movement of the upper housing to the active configuration also vacates the at least one reservoir such that it can receive fluid therein.

In the second active configuration, the upper housing may be supported by an upper portion of the lower housing.

To store the device, the upper housing may be lowered into the lower housing and the two housings secured relative to one another. This may be achieved by clips on at least one sidewall of each housing. The upper housing preferably comprises a handle on an upper surface and the lower housing is preferably mounted on wheels such that the device is readily portable. In addition to assisting portability of the device, the handle allows a user to readily move the upper housing to the second active configuration when the device is required for use.

In a fifth aspect, the present invention provides a waste evacuator device including:

a chamber having an inlet fluidly communicable with an orifice in a mammalian body;

an irrigation member for introducing irrigating fluid into the orifice; and

a suctioning device connected to the chamber by connection means, to create a region of reduced pressure within the chamber;

the device further including a housing having a receiving portion to receive at least part of the chamber and/or the connection means when the device is not in use.

Typically, the receiving portion is a recessed part of the housing that corresponds to the shape and size of the chamber and/or the connection means. The connection means may comprise tubing that connects the suctioning device to the chamber.

The positioning of the chamber and/or the connection means in the receiving portion of the housing allows for easy storage of the components of the device.

In a sixth aspect, the present invention provides a waste evacuator device including:

a chamber having an inlet fluidly communicable with an orifice in a mammalian body;

an irrigation member for introducing irrigating fluid into the orifice; and

a suctioning device connected to the chamber by connection means, to create a region of reduced pressure within the chamber;

the device further including a housing having a receiving portion to receive at least part of the chamber and/or the connection means;

wherein reception of the chamber and/or the connection means by the receiving portion allows the introduction of washing fluid from the irrigation member into at least the chamber.

Typically, the washing fluid is introduced to all surfaces that have come into contact with waste material during the waste evacuation process.

The washing fluid may be the same as the irrigating fluid or, alternatively, it may contain cleaning agents. The cleaning agents are typically not hazardous to a user such that inadvertent use of the washing fluid as irrigating fluid would not be detrimental.

The washing fluid may be drawn from either a multi-functional irrigation reservoir or a separate washing reservoir. Either reservoir typically has the capacity to hold a volume of approximately 5 to 10 litres of fluid for washing.

The washing fluid may be pumped using the irrigation pump and/or the evacuation pump. Typically the pump pressures employed to wash the device are greater than the pressures required for flushing and evacuation.

The inlet of the chamber is typically sealed by the housing when the chamber is received by the receiving portion. The seal formed between the housing and the chamber prevents any leakage of washing fluid during the washing cycle.

In an seventh aspect, the present invention provides a waste evacuator device including:

a chamber having an inlet fluidly communicable with an orifice in a mammalian body;

an irrigation member for introducing irrigating fluid into the orifice for irrigation of a bowel; and

a sectioning device connectable to the chamber to create a region of reduced pressure within the chamber;

wherein the device further includes a flushing means to introduce fluid into the chamber after the introduction of irrigating fluid into the orifice.

The introduction of fluid into the chamber may assist in the evacuation of waste material from the bowel by lubricating the chamber and providing a flow of fluid therefrom.

The flushing means may comprise the irrigation member. In this embodiment the irrigation member is movable relative to the chamber from a position at least partially extending therefrom to enable irrigation of the bowel, to a recessed position wherein it is housed within the chamber. When housed within the chamber, the irrigation member introduces fluid into the chamber during the evacuation process.

In an eighth aspect, the present invention provides a pump member including;

a housing;

at least one conduit extending at least partially through the housing;

one or more roller members positioned within the housing to engage the at least one conduit along a length thereof and so cause any contents of the at least one conduit along that length;

wherein at least the one roller member or one of the roller members is movable relative to the tubing to allow the tubing to be moved relative to the housing.

The pump member may be used with the waste evacuator device of the above aspects. The at least one conduit may be tubing that transports the waste material from the chamber and through the pump member.

The pump member preferably incorporates a plurality of roller members. The roller members may be mounted on a plate like member adapted to rotate within the housing. On rotation of the plate, the roller members compress the tubing that may be mounted between the roller members and an outer wall of the housing. The rotation of the plate is preferably provided by a central or an off centre drive shaft that is rotated by a motor in the housing. The motor is typically powered by a battery but may be mains powered.

In a ninth aspect, the present invention provides a waste evacuator device including:

a chamber having an inlet fluidly communicable with an orifice in a mammalian body;

an irrigation member for introducing irrigating fluid into the orifice; and

a suctioning device connectable to the chamber to create a region of reduced pressure within the chamber; and

a temperature control means to control the temperature of the irrigating fluid and provide irrigating fluid at an optimal temperature to a patient, the temperature control means including;

a temperature sensing means to sense the temperature of the irrigating fluid; and

an actuation means to cause the commencement of introduction of the irrigating fluid into the orifice when the optimal temperature is sensed;

the temperature control means further including heating means to heat the irrigating fluid to the optimal temperature if the temperature sensing means senses that the temperature is below the optimal temperature but within a pre-determined range of the optimal temperature;

the temperature control means further including a means to prevent the introduction of irrigation fluid into the orifice if the temperature sensed is above the optimal temperature and if the temperature is below the optimal temperature and outside the predetermined range of the optimal temperature.

In the embodiment wherein the temperature of the irrigating fluid sensed is above the optimal temperature, the introduction of irrigating fluid into the orifice is prevented until the temperature falls to the optimal temperature.

If the irrigating fluid is too cold and falls outside the pre-determined range, the waste evacuator will not commence irrigation and a user will be required to heat the irrigating fluid to at least within the pre-determined range.

In a tenth aspect, the present invention provides a waste evacuator device including:

a housing;

a chamber having an inlet fluidly communicable with an orifice in a mammalian body;

an irrigation member for introducing irrigating fluid into the orifice; and

a suctioning device connectable to the chamber to create a region of reduced pressure within the chamber; and

a waste outlet connectable to the suctioning device to dispose of waste from the body;

wherein the housing houses the chamber, the irrigation member and the suctioning device therewithin and wherein the housing has a separate receiving portion receive the waste outlet such that the waste outlet is isolated from the chamber, the in irrigation member and the suctioning device during storage of the device.

In an eleventh aspect the present invention provides a waste evacuator device including:

a chamber comprising a body having an inlet fluidly communicable with an orifice in a mammalian body and an outlet;

an irrigation member for introducing irrigating fluid into the orifice for irrigation of a bowel; and

a suctioning device connectable to the chamber to create a region of reduced pressure within the chamber;

wherein when the inlet is in fluid communication with the orifice, the outlet is substantially below the inlet of the chamber and a surface of the body that extends from the inlet to the outlet defines a continuous downward gradient.

When the device of the eleventh aspect is in use, the configuration of the chamber is such that waste material is readily evacuated and less likely to become stuck in the chamber.

Typically, the surface of the body that extends from the inlet to the outlet is positioned substantially adjacent to the body of a user. The surface is at the same level or lower than any part of the body defining the inlet. Accordingly, waste material is forced to move in a downward manner when drawn from the bowel of the user.

The devices, systems and methods of the above aspects of the present invention have particular application in the evacuation of waste product from artificial stomas, such as colostomies. They can, however, also be utilised to gently evacuate waste product from natural stomas, such as the anus.

In a twelfth aspect, the present invention consists in an irrigation cone mountable to, or forming part of, an irrigation source, said irrigation cone including:

an insertion member having a tip portion at least partially insertable into a stoma of a patient and a skirt portion engageable with the skin of a patient around said stoma; and

a pivotable neck member that pivotably supports the insertion member.

The flexible neck member may either be connected to the insertion member or form an integral part of the insertion member.

The irrigation cone may be mounted to, or form an integral part of, an irrigation catheter of a waste evacuation device including the waste evacuation device of the present invention. The irrigation cone may be pivotable relative to the catheter

### BRIEF DESCRIPTION OF THE DRAWINGS

FIG. 1 is a perspective view of the device of the present invention, ready for use;

FIG. 2 depicts the chamber of the device;

FIG. 3 depicts the embodiment of the device of FIG. 1 with the chamber and tubing stored within part of the housing of the device;

FIG. 4 depicts the various components of the device of FIGS. 1 and 3;

FIG. 5 depicts an irrigation cone of the present invention; and

FIG. 6 is a top plan view of the suctioning device of the present invention.

### DETAILED DESCRIPTION OF EXEMPLARY EMBODIMENTS

The waste evacuator device of the present invention is generally depicted as 10 in the accompanying drawings.

The device has a chamber 11, an irrigation member 12 and a suctioning device 13. The suctioning device 13 is connected to the chamber 11 to create a region of reduced pressure within the chamber 11.

The device may, in some embodiments, further comprise an irrigation and evacuation control means which is not depicted in the drawings. The control means is typically a microprocessor that receives signals from sensors positioned within various parts of the device 10. The signals are generally representative of the various functions of the device.

The chamber 11 has an inlet 14 at a first end 15 that is designed to abut with the skin of a patient around an orifice and seal around the orifice.

The chamber 11 also has an outlet 16 at a second end 17. The outlet 16 is connected to the suctioning device 13 by tubing 15. The tubing has a sufficient diameter to accommodate the waste matter drawn from the body.

The chamber 11 of the device is angled along the longitudinal axis such that the outlet 16 is below the inlet 14 when the device 10 is in use. This embodiment uses the force of gravity to aid the removal of waste material from the chamber in addition to suctioning of the waste material from the bowel and through the chamber.

The irrigation member 12 is a catheter 19 that has a lumen (not depicted) extending therethrough for the passage of irrigating fluid. The catheter leas an orifice 21 at a free end 22.

The catheter 19 extends through a sidewall of the chamber 11 and is connected to an irrigation pump 23 via irrigation tubing 24. The free end 22 of the catheter 19 is positioned within the chamber. However, the catheter 19 is movable relative to the chamber 11 such that free end 22 is moveable between a first position within the chamber II and a second position extending beyond the inlet 14 of the chamber 11. When the device 10 is in use and, particularly during the irrigation process, the catheter 19 is moved to the second position such that the free end 22 is insertable into the orifice. This configuration is depicted in FIG. 2.

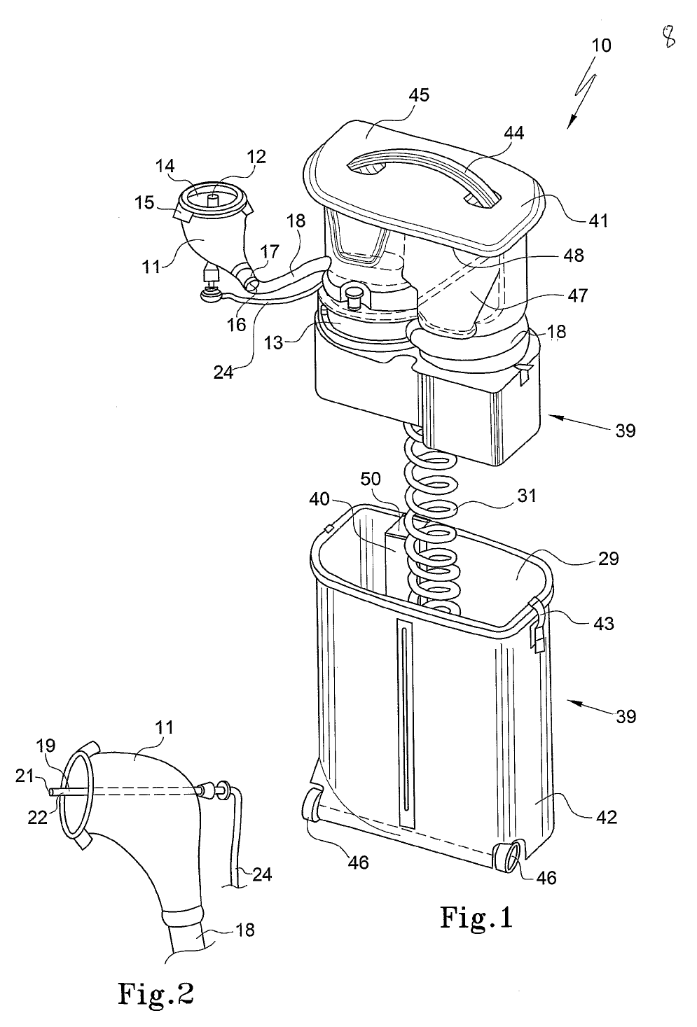

In the first position, that is, after irrigation, the free end 22 of the catheter 19 may be withdrawn back into the chamber 11 as shown in FIG. 1.

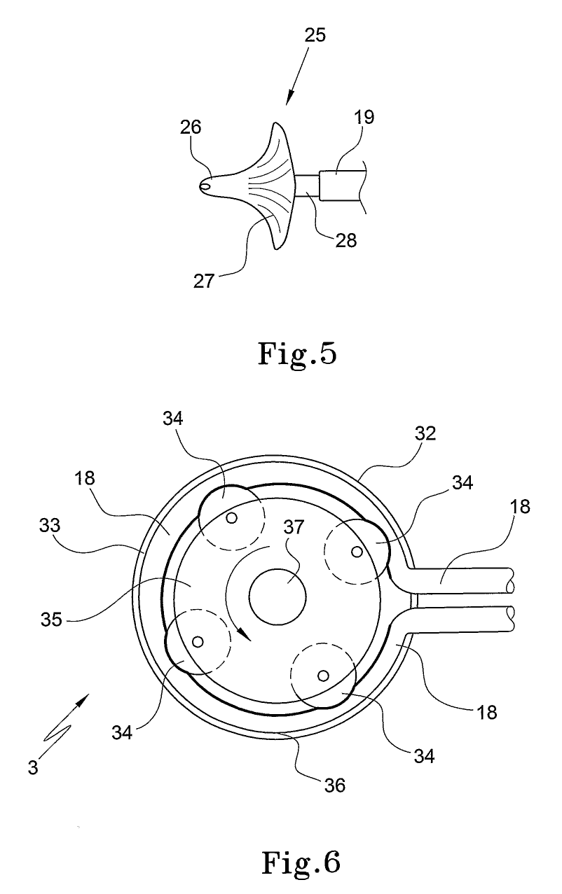

In one embodiment, the catheter 19 includes an irrigation cone 25 at, or adjacent, the free end 22. The irrigation cone 25 is connected to or integral with the catheter 19 as depicted in FIG. 5.

The irrigation cone 25 comprises a tip portion 26 and a skirt portion 27 depending from the tip portion 26. The skirt portion 27 increases in diameter away from the tip portion 26. The skirt portion 27 is engageable with the skin of a patient around a stoma of a user to substantially seal the stoma.

The irrigation cone 25 further includes a neck portion 28 that connects the irrigation cone 25 to the catheter 19. The neck portion 28 is made from a suitably flexible material to allow the irrigation cone 25 to pivot relative to the catheter 19.

Irrigating fluid is forced through the catheter lumen by irrigation pump 23. The irrigation pump further provides a continuous positive pressure in the lumen of the catheter after the irrigation process has ceased. This prevents any waste material entering the lumen during the subsequent evacuation process.

The irrigating fluid is held in reservoir 29 of the device 10. The reservoir 29 depicted in the figures is a multi-functional reservoir and in addition to storing irrigating fluid, also stores fluid to assist in the evacuation cycle and fluid for washing the device after use.

The fluid is drawn from reservoir 29. Various volumes of fluid may be drawn from reservoir 29 for each step of the process, that is, irrigation, evacuation and washing. The volume of fluid drawn from reservoir 29 is controlled by either the control means or the patient.

When the volume of fluid drawn from reservoir 19 is controlled by the control means, the control means receives signals from a pressure sensor in the irrigation reservoir. The signals generated and processed by the control means are indicative of the volume of fluid that has been drawn from the reservoir. When the pressure reaches a pre-determined level, that is, when a pre-determined volume of fluid has been drawn from reservoir 29, the control means sends an output signal to cease the irrigation process.

The suctioning device 13 is a peristaltic-type pump 32. Pump 32 comprises a housing 33 through which tubing 18 passes. Pump 32 also incorporates rollers 34. The rollers 34 are mounted on a disc 35 that rotates within the housing 33. On rotation of the disc 35, the rollers 34 compress the tubing that is mounted between the rollers 34 and an outer wall 36 of the housing 33. The rotation of disc 35 is provided by a central drive shaft 37 that is rotated by a motor (not depicted) in the housing.

During the evacuation of waste product, the irrigation catheter 19 delivers a steady stream of irrigating fluid to the chamber 11 of the device 10. This additional fluid assists in the removal of the waste from the chamber and is drawn from reservoir 29.

The device 10 further provides at least one flush cycle to cause an increased volume of fluid to be delivered to the chamber 11 during evacuation. Again, the fluid for the flush cycle is drawn from reservoir 29.

The device also has a washing function. Fluid is drawn from reservoir 29 pumped around the tubing 18 and 24 using irrigation pump 23 and suctioning device 13.

The device also comprises a heater assembly 38 to warm the fluid in reservoir 29 before it is pumped through the catheter lumen during the irrigation cycle.

The device 10 of the invention includes a housing member 39. When the device 10 is not in use, the chamber 11, the irrigation member 12 and the suctioning device 13 are housed within reservoir 29 of the housing member 39.

The housing member 39 also has a waste outlet tube receiving portion 40. This portion receives the tube that extends from the pump and which is designed to sit within a toilet bowl to allow disposal of the waste material. Because the outlet tube is in contact with the toilet bowl, it is desirable that it is stored separately from the other components of the device. Portion 40 is isolated from reservoir 29 in which the other components may be stored and may be readily accessed by a hinged door 50.

As depicted, the housing member 39 has an upper housing 41 and a lower housing 42 that are connectable to one another by clips 43. The upper housing 41 comprises the chamber 11, the irrigation member 12 and the suctioning device 13. The lower housing 42 comprises reservoir 29.

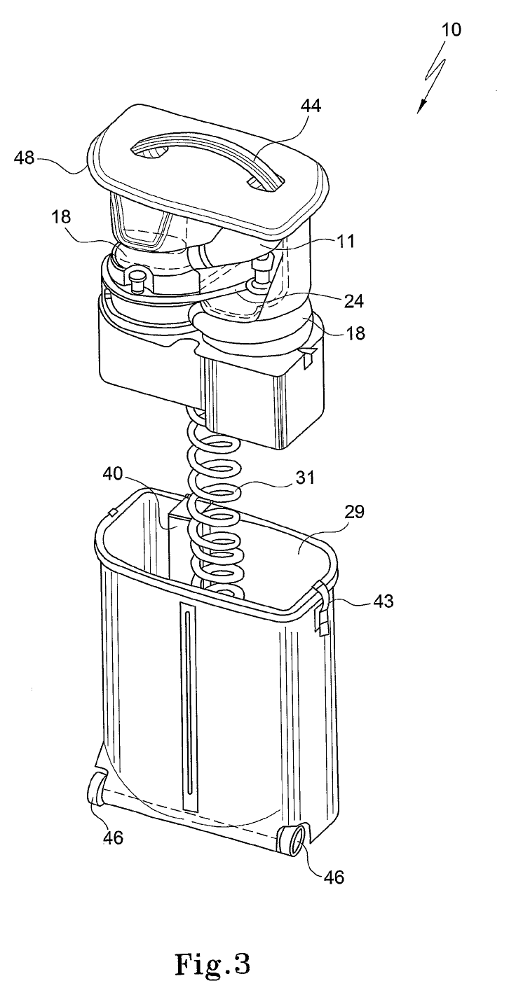

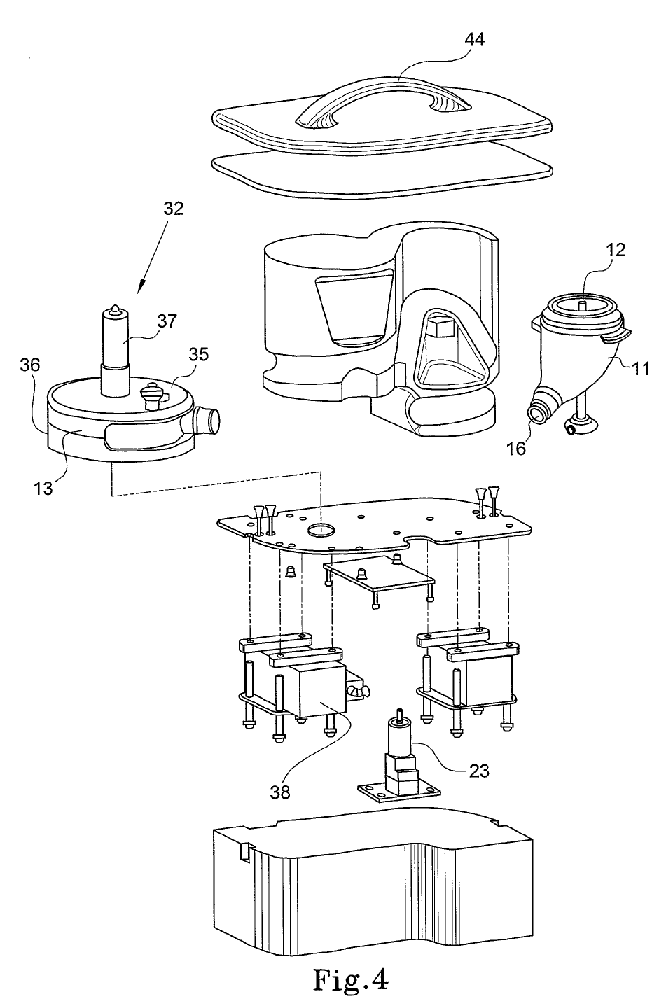

The upper housing 41 is movable relative to the lower housing 42 between a storage configuration wherein the upper housing is substantially housed within the lower housing such that the chamber 11, the irrigation member 12 and the suctioning device 13 are housed within the at least one reservoir 29, and an active configuration (shown in FIGS. 1 and 3) wherein the upper housing 41 extends from the lower housing 42 to expose the chamber 11, the irrigation member 12 and the suctioning device 13 ready for use.

To store the device, the upper housing 41 is lowered into the lower housing 42 and the two housings sewed relative to one another by clips 43. The upper housing 41 has a handle 44 on an upper surface 45 and the lower housing 42 is mounted on wheels 46 such that the device is readily portable. The two housings are biased relative to one another by spring 31.

The housing has a receiving portion 47 to receive the chamber 11 and the tubing 18. The receiving portion 47 comprises a recessed part of the housing that corresponds to the shape and size of the chamber 11 and the tubing 18.

The positioning of the chamber 11 and/or the tubing 18 in the receiving portion of the housing allows for easy storage of the components of the device.

Further, reception of the chamber 11 and the tubing 18 by the receiving portion triggers the washing cycle of the device.

The inlet 14 of the chamber 11 is sealed by a portion 48 of the housing when the chamber 11 is received by the receiving portion. The seal formed between the housing and the chamber prevents any leakage of washing fluid during the washing cycle.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A waste evacuator device comprising:
a chamber having an inlet fluidly communicable with an orifice in a mammalian body;
an irrigation member for introducing irrigating fluid into the orifice;
a suctioning device connectable to the chamber to create a region of reduced pressure within the chamber; and
an irrigation and evacuation control means for the device, said control means receiving signals generated by at least one sensor, said signals being representative of at least both the performance of the irrigation member and of the suctioning device.

2. A device according to claim 1, wherein the pressure and/or flow rate and/or volume of irrigating fluid introduced into the orifice is controlled by said control means.

3. A device according to claim 1, wherein the at least one sensor is positioned in an irrigation reservoir containing said irrigating fluid.

4. A device according to claim 3, wherein the at least one sensor is a pressure sensor which generates signals indicative of the volume of irrigating fluid that has been delivered from said irrigation reservoir into the orifice.

5. A device according to claim 4, wherein upon receiving said signals and determining that a desired volume of irrigating fluid has been delivered into the orifice, the control means ceases emptying said reservoir and thus ceases the irrigation process.

6. A device according to claim 4, wherein the pressure sensor generates signals indicative of the volume of fluid emptied from the reservoir for assisting in evacuating waste from the body and/or for washing the device following use, thereby facilitating control of such evacuating and washing processes by said control means.

7. A device according to claim 1, wherein the control means comprises a micro-processor that receives signals generated from said sensors.

8. A device according to claim 7, wherein the micro-processor processes the signals and generates output signals in response thereto for controlling the irrigation member and/or the suctioning device.

9. A device according to claim 7, wherein said control means further includes one or more manual control systems.

10. A device according to claim 1, wherein the control means receives signals from a pressure sensor positioned in the chamber or tubing connecting the chamber to the suctioning device.

11. A device according to claim 10, wherein in response to said signals being indicative that the region of reduced pressure created by said suctioning device has reached an upper level, the control means generates output signals to either temporarily stop the evacuation or to alter the operation of the suctioning device such that it generates a decreased region of reduced pressure.

12. A device according to claim 1, wherein the commencement, volume of irrigating fluid delivered, and cessation of the irrigating fluid delivery is controlled by the control means.

13. A device according to claim 12, wherein the control means is programmed to control a plurality of fluid delivery cycles during evacuation of waste matter from the body by the suctioning device.

14. A device according to claim 13, wherein following delivery of the irrigating fluid to the chamber, the control means controls the suctioning device to increase the region of reduced pressure within the chamber to assist the removal of the additional irrigating fluid.

15. A device according to claim 1, wherein the control means further controls a washing function wherein washing fluid is drawn from either a multi-functional irrigation reservoir or a separate washing reservoir and circulated through the parts of the device that have come into contact with waste material

16. A device according to claim 15, wherein the control means controls any one or more of; the volume of washing fluid delivered during the washing function, commencement of the washing function, cessation of the washing function, temporary cessation and resumption of the washing function.

17. A system for evacuating waste through an orifice of a mammalian body, the system including:
a chamber having an inlet fluidly communicable with an orifice in a mammalian body;
an irrigation member for introducing irrigating fluid into the orifice;
a suctioning device connectable to the chamber to create a region of reduced pressure within the chamber; and
a control means to at least partially control at least both the introduction of irrigating fluid into the orifice and the reduction of pressure within the chamber.

18. A method for evacuating waste through an orifice in a mammalian body, the method including:
(a) providing a device including a chamber, said chamber having an inlet, the device further including an irrigation member for introducing irrigating fluid into the orifice, a suctioning device connectable to the chamber to create a region of reduced pressure within the chamber; and a control means to control the introduction of irrigating fluid into the orifice and the reduction of pressure within the chamber;
(b) bringing the inlet of the device into fluid communication with the orifice;
(c) introducing irrigating fluid into the orifice through the irrigation member wherein the introduction of the irrigating fluid is at least partially controlled by the control means of the device;
(d) applying suction to draw the waste material from the body and into the chamber of the device wherein the suction is at least partially controlled by the control means of the device.

19. A waste evacuation device including:
a housing having at least one fluid reservoir therein;
a chamber having an inlet fluidly communicable with an orifice in a mammalian body;
an irrigation member for introducing irrigating fluid into the orifice, said irrigation member connectable to the fluid reservoir; and
a suctioning device connectable to the chamber to create a region of reduced pressure within the chamber;
wherein, when the device is not in use, the chamber, the irrigation member and the suctioning device are housed within the fluid reservoir of the housing

20. A device according to claim 19, wherein the at least one fluid reservoir stores irrigating fluid when the device is in use.

21. A device according to claim 20, wherein the chamber, the irrigation member and the suctioning device are housed within at least one of said reservoirs when the device is not in use.

22. A device according to claim 19, further comprising separate upper and a lower housings that are connectable to one another.

23. A device according to claim 22, wherein the upper housing comprises the chamber, the irrigation member and the suctioning device and the lower housing comprises the at least one reservoir.

24. A device according to claim 23, wherein the upper housing is movable relative to the lower housing between a storage configuration wherein the upper housing is substantially housed within the lower housing such that the chamber, the irrigation member and the suctioning device are housed within the at least one reservoir, and an active configuration wherein the upper housing extends from the lower housing to expose the chamber, the irrigation member and the suctioning device ready for use.

25. A device according to claim 24, wherein movement of the upper housing to the active configuration also vacates the at least one reservoir such that it can receive fluid therein.

26. A device according to claim 24, wherein the upper housing is alternatively movable relative to the lower housing to a second active configuration where the upper housing is supported by an upper portion of the lower housing.

27. A device according to claim 26, wherein to store the device, the upper housing is lowered into the lower housing and the two housings are secured relative to one another.

28. A waste evacuator device including:
a chamber having an inlet fluidly communicable with an orifice in a mammalian body;
an irrigation member for introducing irrigating fluid into the orifice; and
a suctioning device connected to the chamber by connection means, to create a region of reduced pressure within the chamber;
the device further including a housing having a receiving portion to receive at least part of the chamber and/or the connection means when the device is not in use.

29. A device according to claim 28, wherein the receiving portion is a recessed part of the housing that corresponds to the shape and size of the chamber and/or the connection means.

30. A device according to claim 28, wherein the connection means comprises tubing that connects the suctioning device to the chamber.

31. A device according to claim 30, wherein the positioning of the chamber and/or the connection means in the receiving portion of the housing allows for easy storage of the components of the device.

32. A waste evacuator device including:
a chamber having an inlet fluidly communicable with an orifice in a mammalian body;
an irrigation member for introducing irrigating fluid into the orifice; and
a suctioning device connected to the chamber by connection means, to create a region of reduced pressure within the chamber;
the device further including a housing having a receiving portion to receive at least part of the chamber and/or the connection means;
wherein reception of the chamber and/or the connection means by the receiving portion allows the introduction of washing fluid from the irrigation member into at least the chamber.

33. A device according to claim 32, wherein the washing fluid is introduced to all surfaces that have come into contact with waste material during the waste evacuation process.

34. A device according to claim 32, wherein the washing fluid is the same as the irrigating fluid or, alternatively, contains cleaning agents.

35. A device according to claim 33, wherein the washing fluid is introduced using the irrigation member and/or the suctioning device.

36. A waste evacuator device including:
a chamber having an inlet fluidly communicable with an orifice in a mammalian body;
an irrigation member for introducing irrigating fluid into the orifice for irrigation of a bowel; and
a suctioning device connectable to the chamber to create a region of reduced pressure within the chamber;
wherein the device further includes a flushing means to introduce fluid into the chamber after the introduction of irrigating fluid into the orifice.

37. A device according to claim 36, wherein the introduction of fluid into the chamber assists in the evacuation of waste material from the bowel by lubricating the chamber and providing a flow of fluid therefrom.

38. A device according to claim 36, wherein the flushing means comprises the irrigation member.

39. A device according to claim 38, wherein the irrigation member is movable relative to the chamber from a position at least partially extending therefrom to enable irrigation of the bowel, to a recessed position wherein it is housed within the chamber.

40. A device according to claim 39, wherein when said irrigation member is housed within the chamber, the irrigation member introduces fluid into the chamber during the evacuation process.

41. A pump member including;
a housing;
at least one conduit extending at least partially through the housing;
one or more roller members positioned within the housing to engage the at least one conduit along a length thereof and so cause any contents of the at least one conduit to move along that length;
wherein at least the one roller member or one of the roller members is movable relative to the conduit to allow the conduit to be moved relative to the housing.

42. A pump member according to claim 41, wherein the roller members are mounted on a plate-like member adapted to rotate within the housing such that upon rotation of the plate-like member, the roller members compress the tubing mounted between the roller members and an outer wall of the housing.

43. A waste evacuator device including:
a chamber having an inlet fluidly communicable with an orifice in a mammalian body;
an irrigation member for introducing irrigating fluid into the orifice; and
a suctioning device connectable to the chamber to create a region of reduced pressure within the chamber; and
a temperature control means to control the temperature of the irrigating fluid and provide irrigating fluid at an optimal temperature to a patient, the temperature control means including;
a temperature sensing means to sense the temperature of the irrigating fluid; and
an actuation means to cause the commencement of introduction of the irrigating fluid into the orifice when the optimal temperature is sensed;
the temperature control means further including heating means to heat the irrigating fluid to the optimal temperature if the temperature sensing means senses that the temperature is below the optimal temperature but within a pre-determined range of the optimal temperature;
the temperature control means further including a means to prevent the introduction of irrigating fluid into the orifice if the temperature sensed is above the optimal temperature and if the temperature is below the optimal temperature and outside the pre-determined range of the optimal temperature.

44. A device according to claim 43, wherein in the event of the temperature of the irrigating fluid sensed being above the optimal temperature, introduction of irrigating fluid into the orifice is prevented until the temperature falls to said optimal temperature.

45. A device according to claim 43, wherein in the event of the temperature of the irrigating fluid sensed being below the optimal temperature, introduction of irrigating fluid into the orifice is presented and said heating means is initiated to heat the irrigating fluid to at least be within said pre-determined range to facilitate resumption of said introduction of said fluid into the orifice.

46. A waste evacuator device including:
a housing;
a chamber having an inlet fluidly communicable with an orifice in a mammalian body;
an irrigation member for introducing irrigating fluid into the orifice; and
a suctioning device connectable to the chamber to create a region of reduced pressure within the chamber; and
a waste outlet connectable to the suctioning device to dispose of waste from the body;
wherein the housing houses the chamber, the irrigation member and the suctioning device therewithin and wherein the housing has a separate receiving portion to receive the waste outlet such that the waste outlet is isolated from the chamber, the irrigation member and the suctioning device during storage of the device.

47. A waste evacuator device including:
a chamber comprising a body having an inlet fluidly communicable with an orifice in a mammalian body and an outlet;
an irrigation member for introducing irrigating fluid into the orifice for irrigation of a bowel; and
a suctioning device connectable to the chamber to create a region of reduced pressure within the chamber;
wherein when the inlet is in fluid communication with the orifice, the outlet is substantially below the inlet of the chamber and a surface of the body that extends from the inlet to the outlet defines a continuous downward gradient.

48. An irrigation cone mountable to, or forming part of, an irrigation source, said irrigation cone including:
an insertion member having a tip portion at least partially insertable into a stoma of a patient and a skirt portion engageable with the skin of a patient around said stoma; and
a pivotable neck member that pivotably supports the insertion member.

49. An irrigation cone according to claim 48, wherein the neck member is flexible and is either connected to the insertion member or forms an integral part of the insertion member.
